(19) United States
(12) Patent Application Publication
(10) Pub. No.: US 2014/0088634 A1
(43) Pub. Date: Mar. 27, 2014

(54) VASCULAR FILTERS AND METHODS FOR USING THEM

(71) Applicant: Archimedes Sanati, Mountain View, CA (US)

(72) Inventor: Archimedes Sanati, Mountain View, CA (US)

(21) Appl. No.: 13/421,808

(22) Filed: Mar. 15, 2012

Related U.S. Application Data
(60) Provisional application No. 61/453,535, filed on Mar. 16, 2011.

(51) Int. Cl. A61F 2/01

(52) U.S. Cl. 606/200

(57) ABSTRACT

Filter devices are provided, as well as apparatus, systems and methods for using such filter devices during a medical procedure within a patient's body. In one embodiment, a system includes a medical device including a proximal end, a distal end sized for introduction into a body lumen, and a filter device including an elongate member that, itself, includes a proximal end, a distal end, and an expandable filter on the distal. One or more features are provided on the elongate member for engaging an outer surface of the medical device, e.g., to slidably or fixedly mount the filter device onto the medical device.

### RELATED APPLICATION DATA

This application claims benefit of co-pending provisional application Ser. No. 61/453,535, filed Mar. 16, 2011, the entire disclosure of which is expressly incorporated by reference herein.

### FIELD OF THE INVENTION

The present invention relates generally to vascular filters, and more particularly to filters that may be deployed within a blood vessel or other body lumen in conjunction with, e.g., mounted on, a catheter, sheath, or other medical device, and to systems and methods for using such filters.

### BACKGROUND

A number of endovascular procedures are presently performed on patients with atherosclerotic and congenital disease and the like to diagnose and/or treat stenotic or occluded regions or other disease types in the patient's blood vessels and/or cavities, such as the aorta, coronary, carotid, cerebral arteries, inferior or superior vena cava, pulmonary arteries, or heart chambers, such as the left ventricle, left atrium, right ventricle, or right atrium. For example, angioplasty may be used to dilate a stenosis, atherectomy may be performed to open severely occluded regions, valvuloplasty may be used to treat valves, or other procedures may be used to implant a device within a blood vessel, heart chamber, or other body lumen. A stent or other prosthesis may be implanted to retain patency of a vessel or valve, either alone or in conjunction with these procedures. In addition, endovascular methods may be used to perform procedures on the heart, such as valve repair, valve replacement, and the like.

One of the problems with these procedures is that embolic material may be released, e.g., from a diseased and/or calcified valve, the wall of a vessel or heart chamber, and the like, during the procedure, and travel downstream where it may become lodged or otherwise cause harm to the patient. For example, ischemic stroke may occur when such emboli are released in the carotid or cerebral arteries and travel to the patient's brain.

To prevent or minimize damage from emboli, vascular filters have been suggested that may be introduced within a blood vessel downstream of a location being treated. Such a filter may be deployed across the vessel to capture embolic material released during a procedure, such as one of the procedures above. Upon completion of the procedure, the filter may be collapsed and removed from the patient.

Therefore, vascular filters that may be used during such procedures would be useful.

### SUMMARY

The present invention is directed generally to vascular filters, and more particularly to filters that may be deployed within a blood vessel, heart chamber, or other body lumen in conjunction with a catheter, sheath, or other medical device, and to systems and methods for using such filters.

In accordance with one embodiment, a system is provided for performing a procedure at a location within a body lumen of a patient that includes a medical device including a proximal end, and a distal end sized for introduction into a body lumen; and a filter device including an elongate member including a proximal end, a distal end, an expandable filter on the distal end of the elongate member, and one or more features on the elongate member for engaging an outer surface of the medical device to mount the filter device onto the medical device.

In an exemplary embodiment, the one or more features may be sized to slidably engage the outer surface of the medical device such that the expandable filter is advanceable along the medical device towards the distal end of the medical device. For example, the one or more features may include one or more collars, e.g., a collar on the distal end of the elongate member adjacent the expandable filter. The one or more collars may include a substantially enclosed ring or may have a “C” shaped cross-section defining opposing edges.

In another embodiment, the elongate member may be a tubular member defining a lumen sized to receive the medical device therein. For example, the tubular member may include a longitudinal slit extending between the proximal and distal ends and defining opposing longitudinal edges, thereby defining the one or more features. The longitudinal edges may be separable to accommodate inserting the medical device laterally between the longitudinal edges such that the medical device is captured within the lumen. Optionally, the tubular member may include lubricious material on an inner surface thereof to facilitate slidably receiving the medical device within the lumen. Alternatively, an inner surface of the tubular member may frictionally engage or otherwise provide an interference fit with the outer surface of the medical device, e.g., to limit axial movement of the tubular member relative to the medical device.

In accordance with another embodiment, a method is provided for performing a procedure at a target location within a patient's body, e.g., using an elongate medical device including proximal and distal ends and one or more diagnostic or treatment element on the distal end. A filter device may be mounted on the medical device such that an expandable filter of the filter device is disposed proximal to the one or more elements, and the medical device with the expandable filter in a collapsed condition may be introduced into the patient's body until the distal end is disposed at a target location within a body lumen. The filter may be deployed such that the filter expands to an enlarged condition at the target location, and a procedure may be performed at the target location using the one or more elements on the medical device, the filter capturing material released during the procedure. For example, the medical device may be a catheter, and the one or more elements may include at least one of a balloon, a stent, and a prosthetic valve.

In an exemplary embodiment, the medical device may be introduced into the patient's body via a sheath, and the sheath may constrain the filter in the collapsed condition. In this embodiment, the filter may be deployed by positioning the filter adjacent a distal end of the sheath and retracting the sheath partially to expose the filter at the target location.

After the procedure, the filter may be collapsed towards the collapsed condition, and the filter device may be removed from the target location, thereby removing the captured material within the collapsed filter. For example, the filter device may be removed simply by removing the medical device from the target location, e.g., if the filter device is substantially fixed relative to the medical device. Alternatively, the filter device may be removed while the distal end of the medical device remains at the target location.

In accordance with still another embodiment, a method is provided for performing a procedure at a target location within a patient's body, e.g., using an elongate medical device including proximal and distal ends and one or more diagnostic or treatment element on the distal end. The distal end of the medical device may be introduced into the patient's body until the distal end is disposed at a target location within a body lumen. A filter device may be advanced over the medical device until an expandable filter on the filter device is disposed proximal to the one or more elements within the target location, and the filter may be deployed such that the filter expands to an enlarged condition at the target location. For example, the medical device may be introduced into the patient's body via a sheath, and the filter device may be advanced over the medical device through the sheath, the sheath constraining the filter in the collapsed condition. The filter may be deployed by positioning the filter adjacent a distal end of the sheath and retracting the sheath partially to expose the filter at the target location.

A procedure may be performed at the target location using the one or more elements on the medical device, the filter capturing material released during the procedure. For example, the medical device may be a catheter, and the one or more elements comprise at least one of a balloon, a stent, and a prosthetic valve. In exemplary embodiments, the procedure may include one or more of angioplasty, stent delivery, valvuloplasty, implanting a valve prosthesis, and atherectomy.

Once the procedure is completed, the filter may be collapsed towards the collapsed condition, and the filter device may be removed from the target location, thereby removing the captured material within the collapsed filter. For example, the filter device may be removed simply by removing the medical device from the target location. Alternatively, the filter device may be removed while the distal end of the medical device remains at the target location. Optionally, thereafter, a second filter device may be advanced over the medical device until an expandable filter on the second filter device is disposed proximal to the one or more elements within the target location.

In another alternative, the distal end of the medical device may be removed from the target location, while leaving the filter device with the filter deployed at the target location. Thereafter, the distal end of a second medical device may be introduced into the patient's body until the distal end is disposed at the target location, e.g., to perform one or more additional procedures.

In accordance with yet another embodiment, a filter device is provided that includes an elongate member including a proximal end, a distal end sized for introduction into a patient's body; an expandable filter on the distal end of the elongate member; and one or more features on the elongate member for engaging an outer surface of a medical device to mount the filter device onto the medical device.

In accordance with still another embodiment, an apparatus is provided for performing a procedure within a patient's body that includes an outer member including a proximal end, a distal end sized for introduction into a patient's body, a lumen extending therebetween, and an expandable filter on the distal end; and a cover member including an inner member slidably received within the outer member lumen, the cover member comprising a proximal end, a distal end, and a cover member on the distal end, the cover member comprising an open proximal end sized for receiving the expandable filter therein in a collapsed condition, the inner member being movable from a proximal position wherein the filter is constrained within the cover, and a distal position wherein the filter is exposed from the cover, the expandable filter biased to expand to an expanded condition when exposed from the cover.

In accordance with yet another embodiment, a method is provided for performing a procedure at a target location within a patient's body using a filter device including an outer member including an expandable filter on a distal end thereof, and an inner member including a cover constraining the filter in a collapsed condition. The distal end of the outer member may be introduced into a patient's body with the expandable filter constrained in the collapsed condition by the cover until the distal end is disposed at a target location within a body lumen. The inner member may be advanced relative to the outer member to expose the filter such that the filter expands to an enlarged condition at the target location, and a procedure performed at the target location, the filter capturing material released during the procedure.

Other aspects and features of the need for and use of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a perspective view of a first embodiment of a filter device that includes an elongate wire including an expandable filter and a collar on a distal end of the wire for placing the filter device around a catheter or other tubular device.

FIG. 2 is a perspective view of a second embodiment of a filter device that includes an elongate wire including an expandable filter on a distal end of the wire and a plurality of collars for placing the filter device around a catheter or other tubular device.

FIG. 3 is a perspective view of a third embodiment of a filter device that includes an elongate tubular body including an expandable filter on a distal end of the wire, the tubular body including a longitudinal slit for placing the filter device around a catheter or other tubular device.

FIGS. 4A and 4B are side views of a distal end of a system including a filter device, such as those shown in FIGS. 1-3, mounted on a balloon catheter with an expandable filter positioned within a guide catheter or sheath in a collapsed condition and deployed from the sheath in an enlarged condition, respectively.

FIG. 5 is a cross-sectional view of a patient's body, showing the system of FIGS. 4A and 4B being introduced into the patient's aorta and heart during a procedure.

FIGS. 6A and 6B are side views of a distal end of an exemplary embodiment of a guide catheter that includes a filter device including an expandable filter and a cover that may be removed from the filter to expand the filter from a collapsed condition to an enlarged condition, respectively.

### DETAILED DESCRIPTION

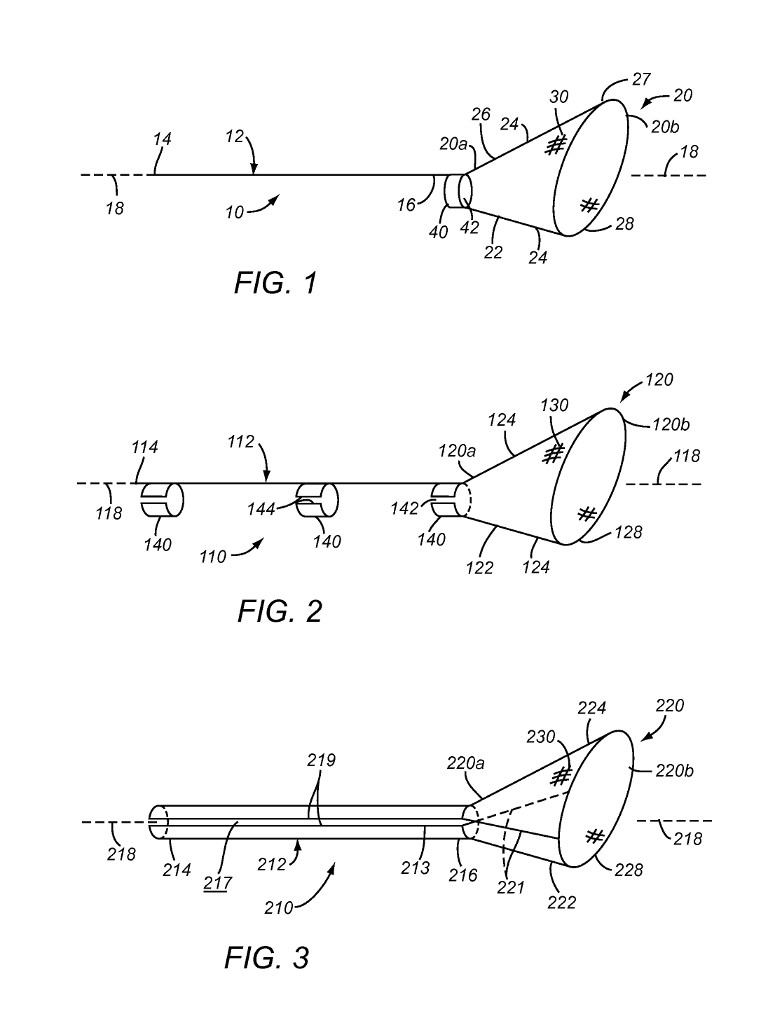

Turning to the drawings, FIG. 1 shows an exemplary embodiment of a filter device 10, which generally includes a wire or other elongate member 12 including a proximal end 14 and a distal end 16 defining a longitudinal axis 18 therebetween. An expandable filter 20 may be carried on the distal end 16, and one or more features may be provided on the distal end 16 for mounting the filter device 10 onto an elongate medical device (not shown), e.g., a collar 40, as described further below.

The wire 12 may be similar to a conventional guide wire or other rail, e.g., a hollow or solid member, having a length similar to that of a medical device to which the filter device 10 may be mounted, e.g., between about twenty and one hundred fifty centimeters (20-150 cm). For example, the wire 12 may have sufficient length to be introduced into a desired location within a patient's body, e.g., within the patient's vasculature, from a remote access site, e.g., a percutaneous access site, such as the femoral artery, carotid artery, radial artery, brachial artery, and the like (not shown). Alternatively, as described further below, the wire 12 may have a length substantially shorter than the medical device to which the filter device 10 may be mounted, e.g., if the filter device 10 is substantially fixed axially once mounted on the medical device.

If the filter device 10 is movable relative to the medical device on which it is mounted, the wire 12 may have sufficient column strength to advance the distal end 16 by pushing or otherwise manipulating the wire 12 from the proximal end 14 without substantial risk of the wire 12 buckling or kinking. In exemplary embodiments, the wire 12 may be a helically wound wire formed from metal such as stainless steel, optionally coated or including desired material, e.g., PTFE or other lubricious material, to facilitate advancing the wire 12 along another device, as described further below. The wire 12 may have a substantially uniform or variable diameter or other cross-section along its length, e.g., not more than about 0.038 inch (1 mm).

The collar 40 may be attached or otherwise fixed on the distal end 16, e.g., adjacent to the filter 20. The collar 40 may be a substantially enclosed ring, e.g., defining an axial opening 42 sized to receive a catheter or other elongate medical device (not shown) therethrough, as described further below. The collar 40 may be substantially rigid, e.g., such that the opening 42 has a fixed diameter. Alternatively, the collar 40 may be flexible or semi-rigid, e.g., may be elastically expandable such that the collar 40 may be expanded, e.g., to accommodate receiving a medical device through the opening 42 that is slightly larger than the diameter of the collar 40 in a relaxed state.

Alternatively, the collar 40 may have a “C” shaped cross-section, similar to the collars 140 of the filter device 110 of FIG. 2 described further below. In this alternative, the collar 40 may be opened to place the filter 20 around a catheter or other tubular device, e.g., to allow the filter device 10 to be mounted laterally around a medical device, rather than from one end of the medical device, also as described further below.

With continued reference to FIG. 1, the filter 20 includes a substantially closed first end 20a attached to the distal end 16 of the wire 12 and/or to the collar 18, and an open second end 20b. In this embodiment, the open end 20b is disposed distally to the closed end 20a, although, it will be appreciated that the orientation of the filter 20 may be reversed, if desired, e.g., if the filter is intended to be disposed distally to a treatment element on the medical device rather than proximally to the treatment element, e.g., if the filter device 10 is introduced in a body lumen in the same direction as flow within the body lumen.

As shown, the filter 20 may include an expandable frame 22 and filter material 30 attached to the frame 22. For example, the frame 22 may include a plurality of generally longitudinal struts, splines, or other supports 24 extending at least partially between the first and second ends 20a, 20b, e.g., having proximal ends 26 attached to distal end 16 of the wire 12 and/or to the collar 18, and distal ends 27 terminating adjacent the open end 20b. Optionally, the frame 22 may include a loop or other support 28 extending around the open end 20b, e.g., if desired to bias the open end 20b towards a predetermined shape, e.g., a circular or elliptical shape. The supports 24, 28 may be attached at one or more regions to the filter material 30, e.g., by bonding with adhesive, fusing, sonic welding, and the like, such that the frame 22 generally supports the filter material 30.

In one embodiment, the supports 24, 28 of the frame 22 may be biased to expand towards an enlarged condition, yet compressible inwardly towards a collapsed condition. For example, as shown in FIG. 1, the frame 22 may be biased to a generally conical, funnel, or other tapered shape between the open end 20b and the closed end 20a. During use, the frame 22 may be compressed inwardly, e.g., around an elongate medical device (not shown) over which the collar 40 has been placed and/or within a sheath, as described further below. For example, the longitudinal struts 24 may be compressed into a substantially axial orientation or wound into a compressed helical orientation around the medical device, thereby folding or winding the filter material 30 in the collapsed condition.

When not constrained in the collapsed condition, the supports 24, 28 may automatically expand towards the enlarged condition, causing the filter material 30 to open and assume the tapered shape shown in FIG. 1. For example, the supports 24, 28 may be formed from round or flat band wire elements, e.g., from elastic or superelastic material, such as Nitinol, that may be elastically compressed towards the collapsed condition yet resiliently expand towards the enlarged condition.

Alternatively, the frame 22 may be sufficiently flexible that the filter 20 may be reversed and compressed around the medical device with the open end 20b disposed proximal to the closed end 20a. For example, the longitudinal struts 24 may be folded proximally and then compressed around the medical device, which may facilitate advancing the filter in the collapsed condition through a guide catheter or other sheath (not shown). In this alternative, the longitudinal struts 24 may include hinged regions or may be sufficiently flexible at intermediate locations between the ends 26, 27 to accommodate the struts 24 bending to reorient the filter 20 from the proximal orientation back to the distal orientation. For example, after the filter 20 is exposed within a body lumen, the guide catheter or sheath from which the filter 20 is deployed may be advanced to push the filter 20 from the proximal orientation to the distal orientation, thereby orienting the filter upstream within the body lumen (assuming the filter device 10 is introduced in a retrograde manner against the flow within the body lumen).

Additional information on struts or frames that may be provided on the filter 20 are disclosed in U.S. Pat. No. 6,027,520, the entire disclosure of which is expressly incorporated by reference herein.

The filter material 30 may be a woven fabric, wire frame, or other known structure that may be attached to the frame 22, to the collar 40, and/or to the distal end 16 of the wire 12. For example, the filter material 30 may be formed from a braided or otherwise woven mesh, or a polymeric sheet with holes provided therein, similar to materials in the references incorporated by reference elsewhere herein. In an exemplary embodiment, one end of the filter material 30 may be attached to the collar 40 to substantially enclose the closed end 20a. The filter material 30 may extend along and/or around the struts 24 to the open end 20b, thereby defining a pocket within the filter material 30 for capturing embolic material. The open end 20b of the filter material 30 may be self-supporting or may include the loop support 28 to bias the open end 20b to a desired shape. When the frame 22 is directed back towards the collapsed condition, e.g., after deploying the filter 20 within a body lumen, the open end 20b of the filter material 30 may be substantially closed such that any embolic material captured in the pocket is trapped within the filter 20, e.g., for removal from the patient's body, as described elsewhere herein.

Turning to FIG. 2, in another embodiment, a filter device 110 is shown that includes a wire or other elongate member 112 including a proximal end 114 and a distal end 116 defining a longitudinal axis 118 therebetween, and an expandable filter 120 carried by the distal end 116, e.g., similar to the previous embodiment. However, unlike the previous embodiment, the filter device 110 includes a plurality of collars 140 spaced apart along a length of the wire 112. For example, as shown, a collar 140 is provided on the distal end 116 adjacent the filter 120, another is provided at an intermediate location, and another is provided adjacent the proximal end 114 of the wire 112. It will be appreciated that one or more additional or fewer collars (not shown) may be provided along the length of thee wire 112, as desired.

Each collar 140 has a “C” shaped cross-section defining an axial opening 142 therethrough, although, alternatively, each collar may define a substantially enclosed ring, similar to the collar 40 described above. As shown, opposing edges 144 of the collars may be spaced apart from one another in a relaxed condition such that the edges 144 may be opened or separated, e.g., to accommodate receiving a catheter or other elongate medical device laterally between the edges 144 into the openings 142. For example, each collar 140 may define a perimeter having an arc of greater than one hundred eighty degrees (180°), such that the opposing edges 144 are spaced apart by less than one hundred eighty degrees (180°), and the collar 140 may be securely received around the medical device.

The openings 142 may be sized to slidably receive the medical device therethrough, e.g., such that the filter device 110 may be placed around the medical device and then advanced or retracted axially along the medical device, as described further below. For example, the edges 144 may be separable to accommodate inserting the medical device laterally between the edges 144 such that the medical device is captured within the openings 142. Optionally, a lubricious coating or other material may be provided on an inner surface of the collars 140, e.g., to facilitate slidably receiving the medical device within the openings 142.

In an alternative embodiment, the collars 140 may define an inner diameter in a relaxed state that is smaller than an outer diameter of the medical device such that an inner surface of the collars 140 frictionally engages the outer surface of the medical device or otherwise provides an interference fit, e.g., to limit axial movement of the filter device 110 relative to the medical device. In this alternative, the interference fit between the collars 140 and the outer surface of the medical device, e.g., due to the bias of the collars 140 to return to their relaxed state, may be sufficient to substantially fix the collars 140 axially and/or rotationally relative to the medical device. Consequently, the collars 140 may substantially fix the filter 120 relative to the medical device. Optionally, friction-enhancing surfaces, materials, and/or other features may be provided on the collars 140, if desired to ensure to undesired movement once the filter device 110 is mounted on the medical device.

Turning to FIG. 3, yet another embodiment of a filter device 210 is shown that includes an elongate tubular member 212 including a proximal end 214, a distal end 216, and a lumen 217 extending therebetween generally defining a longitudinal axis 218. An expandable filter 220 may be carried by the distal end 216, which may be similar to any of the embodiments described herein.

As shown, the tubular member 212 includes a longitudinal slit 213 extending between the proximal and distal ends 214, 216 and defining opposing longitudinal edges 219. Similar to the collars 140 described above, the longitudinal edges 219 may be separable to accommodate inserting a catheter or other medical device laterally between the longitudinal edges 219 such that the medical device is captured within the lumen 217. Alternatively, the tubular member 212 may be substantially enclosed, i.e., without the longitudinal slit 213 to define an enclosed lumen 217. In this alternative, the tubular member 212 may have a substantially fixed inner diameter or may be flexible or otherwise expandable, e.g., to accommodate receiving various size medical devices, which may be slightly larger than the lumen 217 when the tubular member 212 is in a relaxed state.

Optionally, the tubular member 212 may include a lubricious coating or material on an inner surface thereof, e.g., to facilitate slidably receiving a medical device within the lumen 217. Alternatively, the tubular member 212 may have an inner diameter in a relaxed state that is smaller than an outer diameter of the medical device onto which the filter device 210 is to be mounted such that an inner surface of the tubular member 212 frictionally engages the outer surface of the medical device or otherwise provides an interference fit to limit axial movement of the tubular member 212 relative to the medical device.

The filter 220 may define a substantially enclosed perimeter between the open and closed ends 220b, 220a. Alternatively, as shown, the filter 220 may have one or more longitudinal seams 221 extending between the open and closed ends 220b, 220a. For example, the longitudinal seam 221 may be aligned with the slit 213 such that the longitudinal seam 221 may be opened when the slit 213 is opened, e.g., to facilitate mounting the filter 220 laterally around an elongate medical device when the tubular member 212 is placed around the medical device. The filter material 230 may include edges that overlap, abut, or are spaced apart slightly from one another in the enlarged condition, e.g., to minimize any gap that may otherwise allow material to escape past the filter material 230 during use. For example, during use, the longitudinal seam 221 may be opened to accommodate placing the filter 220 around a medical device being inserted into the lumen 216. After the filter device 210 is mounted on the medical device, the longitudinal seam 221 may be released, and the frame 222 may bias the filter 220 to assume a substantially enclosed configuration, as shown in FIG. 3.

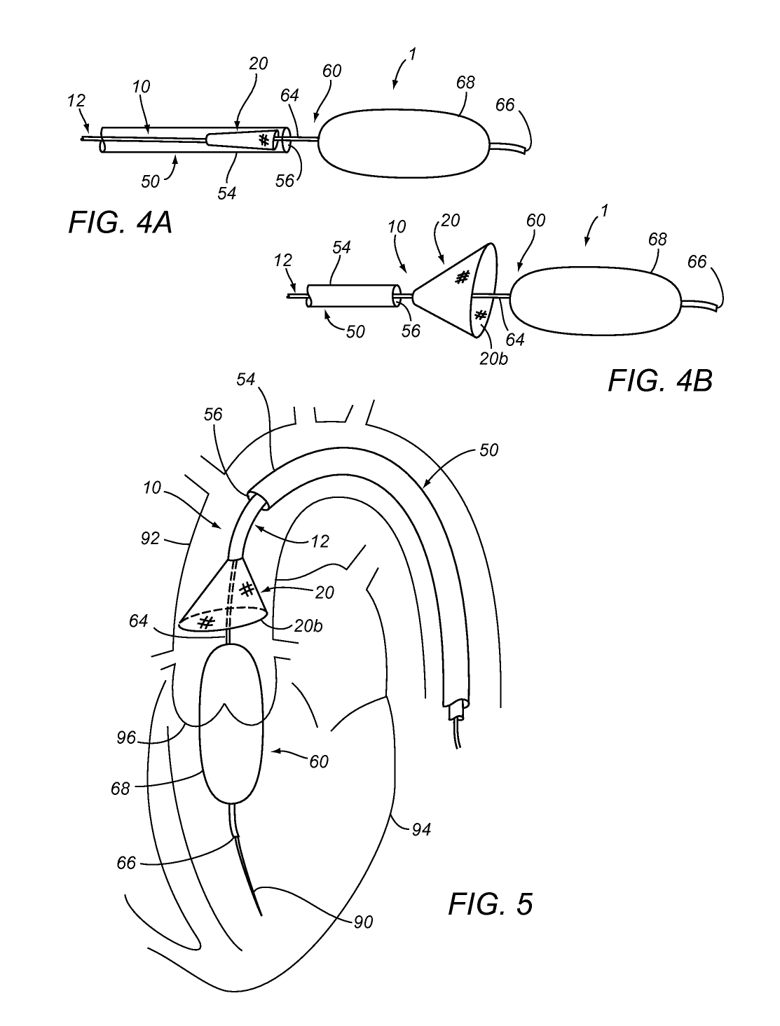

Turning to FIGS. 4A and 4B, any of the filter devices herein 10, 110, 210 (although references below are generally to the filter device 10 merely as an example) may be provided as part of a system 1, e.g., including a tubular sheath, sleeve, guide catheter, or other constraint 50, and, optionally, a medical device, such as catheter 60. Generally, the sheath 50 may include a proximal end (not shown), a distal end 54, and a lumen 56 extending therebetween. The lumen 56 may be sized to receive one or more instruments therethrough, e.g., the catheter 60 with a filter device 10 mounted or being loaded thereon. For example, the catheter 60, with the filter device 10 mounted thereon outside a patient's body, may be introduced into the proximal end of the sheath 50, e.g., after compressing the filter 20 to its collapsed condition, as shown in FIG. 4A. Alternatively, the filter device 10 may be mounted on the proximal end of the catheter 60 while the distal end 64 is disposed within the patient's body, and advanced through the sheath 50 to the distal end 64, as described further below. When the filter 20 is exposed beyond the distal end 54 of the sheath 50, the filter 20 may resiliently expand towards the enlarged condition, as shown in FIG. 4B, and as described further elsewhere herein.

In an exemplary embodiment, the sheath 50 may simply be a conventional guide catheter used during endovascular procedures. Alternatively, the sheath 50 may be a specialized sheath configured for covering and/or uncovering the expandable filter 20. In this alternative, the sheath 50 may be provided to the user as part of the filter device 10, e.g., with the distal end 54 disposed over the filter 20 (thereby constraining the filter 20 in the collapsed condition), or with the distal end 54 disposed adjacent the filter 20 (with the filter 20 in the enlarged condition, such that the sheath 50 may be advanced over the filter 20 immediately before a procedure).

The catheter 60 may be any tubular or other elongate medical device intended for performing a therapeutic and/or diagnostic procedure within a patient's body. Generally, the catheter 60 includes a proximal end (not shown), a distal end 64 sized for introduction into a body lumen, and one or more diagnostic and/or treatment elements on the distal end 64. For example, as shown, the element may be a balloon 68, e.g., a non-compliant angioplasty balloon. In addition or alternatively, other diagnostic and/or treatment elements may be provided on the distal end 64, such as a heart valve, a stent, an atherectomy device, and the like (not shown). Such elements may result in the release of embolic or other material within the body lumen being treated during their use, and, therefore, may benefit from use of the filter 20, as described further below. Optionally, the catheter 60 may include one or more lumens extending between the proximal end and the distal end 64, e.g., a guidewire lumen 66, similar to other conventional endovascular devices.

Turning to FIG. 5, a system 1 including any of the filter devices herein (e.g., filter 10) may be used to perform a procedure within a body lumen of a patient's body. For example, as shown, the system 1 may be used for performing a procedure within the aorta 92 and/or heart 94 of a patient. It will be appreciated that the system 1 may be used in a variety of locations and/or procedures within a patient's body, e.g., within the patient's vasculature, as described elsewhere herein.

Once a procedure and any instruments are identified, a filter device 10 may be selected that is appropriate for being used in conjunction with one or more of the instruments. For example, if a balloon catheter 60 is to be used, e.g., to dilate valve leaflets, a stenosis, or other obstruction within the patient's body, a filter device 10 may be selected that is sized to be mounted on the catheter 60, thereby providing a filter functionality to the catheter 60 without having to provide a specialized catheter.

In one embodiment, the filter device 10 may be mounted on the catheter 60 outside the patient's body. For example, if the filter device includes one or more “C” shaped collars 140 (as in the filter device 110 of FIG. 2) or includes a slitted tubular member 212 (as in the filter device 210 of FIG. 3), the filter device may be loaded laterally onto the catheter 60. Alternatively, if the collars or tubular member are substantially enclosed (not shown), the filter device may be loaded longitudinally over the catheter 60, e.g., from the distal end 64 or the proximal end.

As shown in FIGS. 4A and 4B, the filter device 10 may be mounted on the catheter 60 such that the filter 20 is located adjacent the balloon 68, e.g., offset proximally from the balloon 68 by a desired distance. This configuration may substantially fix the filter device 10 on to the catheter 60, e.g., such that the result is a catheter 60 including a fixed filter 20 thereon. Optionally, in this configuration, the length of the wire 12 of the filter device 10 may be substantially shorter than the catheter 60, e.g., such that the wire 12 extends proximally only partially from the distal end 64 of the catheter 60.

Alternatively, the filter device 10 may be slidable relative to the catheter 60, e.g., such that the filter device 10 may be advanced with the catheter 60, yet may be withdrawn or otherwise manipulated independently of the catheter 60, if desired. In yet another alternative, the filter device 10 may be mounted to the catheter 60 after introducing the catheter 60 into the patient's vasculature, as described further below.

In an exemplary procedure, a guidewire 90 may be placed at the target location, such as the ascending aorta 92, e.g., from an entry site, such as a percutaneous entry site in a femoral artery, carotid artery, radial artery, brachial artery, and the like. Optionally, a guide catheter 50 may also be placed at the target location, e.g., over or otherwise in conjunction with the guidewire 90. With the filter 20 compressed in the collapsed condition (e.g., by the guide catheter 50 itself, or by an optional constraining sheath, not shown), the distal end 64 of the catheter 60 and the filter device 10 may be introduced into the lumen 56 of the guide catheter 50 and/or over the guidewire 90 to the target location, e.g., as shown in FIG. 4A.

When desired, the guide catheter 50 may be retracted or the catheter 60 and filter device 10 may be advanced to deploy the filter 20 within the aorta 92 or other body lumen. For example, the filter 20 may be oriented distally and simply expand to open the open end 20b as the filter 20 is deployed from the distal end 54 of the guide catheter 50. Alternatively, if the filter 20 is oriented proximally, the guide catheter 50 may be retracted sufficient distance to completely release the filter 20 within the aorta 92. The guide catheter 50 may then be advanced such that the distal end 54 contacts and pushes the filter 20, thereby reorienting the filter 20 from the proximal orientation to the distal orientation (not shown). In a further alternative, if the frame 22 is sufficiently flexible and biased towards the distal orientation, the filter 20 may unfold and automatically reorient itself distally when released.

Consequently, the filter 20 may automatically expand and/or extend across the aorta 92, as shown in FIGS. 4B and 5. Once fully deployed, the balloon 68 may be inflated, e.g., to treat leaflets 96 of the heart 94 or otherwise perform one or more desired procedures within the aorta 92 or other target location. After completing the procedure(s), the catheter 60 and filter device 10 may be retracted back into the guide catheter 50. As the closed end 20a of the filter 20 enters the lumen 56 of the guide catheter 50, the distal end 54 of the guide catheter 50 may cause the filter 20 to compress inwardly towards the collapsed condition, thereby trapping any material captured in the filter 20 and withdrawing the material into the guide catheter 50. The catheter 60 and filter device 10 may then be removed entirely from the patient's body, and thereafter the guide catheter 50 may be removed.

Alternatively, if the filter device 10 is movable relative to the catheter 60, the catheter 60 may be manipulated within the aorta 92 (or other body lumen) during treatment, while maintaining the filter 20 deployed and in position, e.g., substantially stationary. In addition, if desired, the filter device 10 may be withdrawn into the guide catheter 50 without the catheter 60, e.g., if it is desired to remove material captured by the filter 20. Optionally, another filter device (or the same filter device after cleaning or removal of captured material within the filter) may then be advanced over the catheter 60 via the guide catheter 50 (which may constrain the filter 20 of the new filter device in the collapsed condition), and deployed within the aorta 92, if it is desired to capture additional material being released within the aorta 92.

In another alternative, the catheter 60 may be removed, while leaving the filter device 10 deployed within the aorta 92. For example, in this alternative, one or more other instruments, e.g., different catheters, sheaths, or devices (not shown) may be introduced through the guide catheter 50 and/or over the guidewire 90 such that the device(s) pass through the collar 40 of the filter device 10, thereby providing substantially continuous filtering while multiple procedures are completed at the target location with different instruments.

In yet a further alternative, the catheter 60 may be introduced via the guide catheter 50 into the aorta 92 before or during a procedure without a filter device being mounted on the catheter 60 or already placed through the guide catheter 50. In this alternative, at any point thereafter, a filter device 10 (or a series of filter devices, if desired) may be mounted on the proximal end of the catheter 60 and introduced via the guide catheter 50 to deploy a filter 20 within the aorta 92, e.g., before performing the procedure with the balloon 68 within the aorta 92.

For example, if the filter device 10 includes “C” shapes features, the filter device 10 may be mounted laterally over the proximal end of the catheter 60. If the filter device 10 includes enclosed features, e.g., a collar 40 within a substantially enclosed opening 42 or a tubular member 212 including an enclosed lumen 216, the filter device 10 may be inserted over the proximal end of the catheter 60. In this configuration, the filter device 10 may also be directed over the distal end 64 of the catheter 60 before introducing the catheter 60 into guide catheter 50.

Once the one or more procedures are completed, the filter device 10, catheter, 60, guidewire 90, and guide catheter 50 may be removed sequentially or simultaneously, as desired.

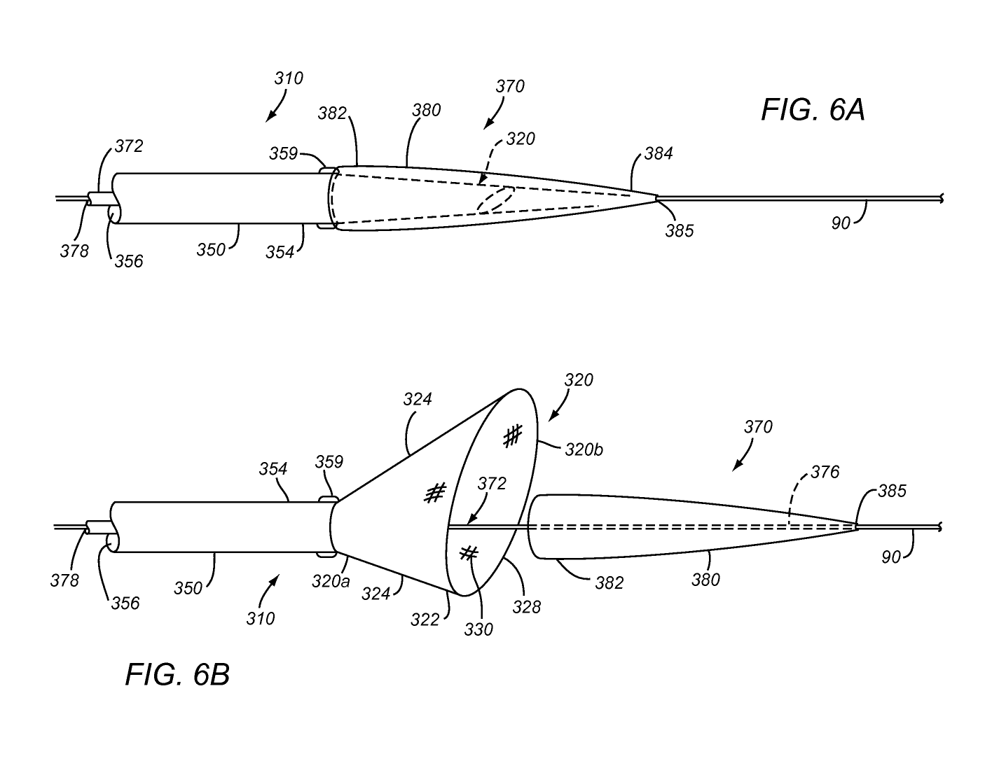

Turning to FIGS. 6A and 6B, another embodiment of a filter device 310 is shown that includes an elongate guide catheter or other outer sheath 350 including a proximal end (not shown), a distal end 354, and a lumen 356 extending therebetween. A filter 320 is carried on the distal end 354, which may be expandable from a collapsed condition (shown in FIG. 6A) to an enlarged condition (shown in FIG. 6B), similar to other embodiments herein. Also similar to previous embodiments, the filter 320 may include a frame 322, e.g., including struts 24, 28, and filter material 330, thereby defining a closed first end 320a and an open second end 320b. The closed end 320a may be attached to the distal end 354 of the sheath 350, e.g., around an inner surface and/or an outer surface thereof, for example, by bonding with adhesive, fusing, sonic welding, and the like.

Unlike the previous embodiments, the filter device 310 also includes a cover member 370, which may be used to selectively cover the filter 320 and constrain the filter 320 in the collapsed condition. Generally, the cover member 370 includes an inner sheath, wire, shaft, or other elongate member 372 including a proximal end (not shown), a distal end 376 sized for introduction through the lumen 356 of the sheath 350, and, optionally, a guidewire lumen 378 extending therebetween such that the filter device 310 may be introduced over a guidewire 90, as described further below.

A cover 380 is attached to the inner member 372 that includes an open first or proximal end 382 and a second or distal end 384. The open end 382 of the cover 380 may be sized to be received over the filter 320 when the filter 320 is compressed to the collapsed condition, e.g., to constrain the filter in the collapsed condition. The open end 382 may have a substantially fixed diameter or may be capable of being opened or closed, e.g., to facilitate in compressing the filter 320 or retracting the cover 380 into the outer sheath 350, as described further below. The second end 384 of the cover 380 may be closed and/or may tapered, e.g., to provide a substantially atraumatic distal tip for the device 310. In an exemplary embodiment, the cover 380 may be formed from sufficiently flexible material to provide a flexible atraumatic tip, which may facilitate advancement of the filter device 310 through tortuous anatomy. Optionally, if the inner member 372 includes a guidewire lumen 378, the second end 384 of the cover 380 may include an opening 385 therein, e.g., communicating with the guidewire lumen 378 or through which the distal end 376 of the inner member 372 may extend.

Optionally, the sheath 350 may include a seal or other transition feature 359 on the distal end 354, which may slidably engage the proximal end 382 of the cover 380, e.g., to provide a substantially smooth transition between the cover 380 and the sheath 350. Alternatively, if the filter 320 is attached to the inner surface of the sheath 350, the proximal end 382 of the cover 380 may be sized to be received partially within the distal end 354 of the sheath 350 to provide a substantially smooth transition.

In one embodiment, the filter device 310 may be used in place of a conventional sheath or guide catheter, e.g., such that the filter device 310 may be used to deliver one or more instruments, such as one or more catheters (not shown), similar to the devices and methods described above. Thus, the device 310 may provide functionality of a conventional guide catheter, i.e., facilitating introduction of one or more instruments into a target location, while also providing filtering at the target location in the event embolic material is released by the instrument(s). In another embodiment, the filter device 310 may be introduced from a different location than any other instruments used during a procedure, e.g., advanced through another blood vessel or body lumen different from that used for the other instruments, yet deployed adjacent the target location to provide filtering. In still another alternative, the filter device 310 may be used as a stand-alone filtering system, e.g., delivered in the ascending aorta or other vessel downstream of a diseased region, such as a diseased valve or other structure that is being treated via another approach, such as a transapical, transatrial, or septal approach.

Before use, the filter device 310 may be provided with the filter 320 in the collapsed condition and the cover 380 positioned over the filter 320, as shown in FIG. 6A. For example, during manufacturing or immediately before use, the inner member 372 may be inserted through the lumen 356 of the guide catheter 350 until the cover 380 is disposed distally beyond the filter 320, as shown in FIG. 6B. The filter 320 may then be rolled or otherwise compressed to the collapsed condition, and then the inner member 372 may be retracted sufficiently to insert the filter 320 into the open end 382 of the cover 380. The filter 320 may then be released within the cover 380, which may constrain the filter 320 in the collapsed condition.

Thereafter, during use, with the cover 380 provided over the filter 320 as shown in FIG. 6A, the sheath 350 and inner member 372 may be advanced into a patient's body, e.g., through the patient's vasculature, into a target location. For example, a guidewire or other rail 90 may be previously placed from an entry site to the target location, e.g., similar to other embodiments herein, and the guidewire 90 may be backloaded through the opening 385 in the cover 380 and/or the lumen 378 of the inner member 372. The device 310 may then be advanced over the guidewire 90, e.g., until the filter 320 and cover 380 are positioned at the target location. Once positioned as desired, the cover member 370 may be advanced relative to the sheath 350 to expose the filter 320 from the cover 380, as shown in FIG. 6B. The filter 320 may automatically expand towards the enlarged condition, as shown, thereby deploying a filter within the target location.

If the cover 380 is smaller than the lumen 356 of the sheath 350, the cover member 370 may be withdrawn from the target location through the sheath 350. Alternatively, the cover 380 may be collapsible, e.g., from the proximal end of the cover member 370, and may be collapsed and removed via the sheath 350. In a further alternative, the cover member 370 may simply be directed away from the distal end 354 of the sheath 350 such that the cover 380 is located away from the target location.

Once the filter 320 is deployed, one or more instruments (not shown) may be introduced into the target location, e.g., via the lumen 356 of the sheath 350 and/or over the guidewire 90 to perform one or more diagnostic and/or therapeutic procedures, such as those described elsewhere herein. Upon completing the procedure, any instruments may be removed, and a secondary sheath or sleeve may be advanced over the sheath 350, e.g., to compress and/or otherwise capture the filter 320. For example, the secondary sleeve may be introduced from the proximal end over the sheath 350 until a distal end of the sleeve is disposed around the distal end 354 proximal to the filter 320. The sheath 350 may then be withdrawn, pulling the filter into the secondary sleeve, thereby compressing the filter 320 back towards the collapsed condition and capturing material within the filter. The sheath 350 and secondary sleeve may then be removed.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A system for performing a procedure at a location within a body lumen of a patient, comprising:
a medical device including a proximal end, and a distal end sized for introduction into the body lumen; and
a filter device including an elongate member including a proximal end, a distal end, an expandable filter on the distal end of the elongate member, and one or more features on the elongate member for engaging an outer surface of the medical device to mount the filter device onto the medical device to substantially fix the filter device axially relative to the medical device.

2. (canceled)

3. The system of claim 1, wherein the one or more features comprise a collar on the distal end of the elongate member adjacent the expandable filter.

4-9. (canceled)

10. The system of claim 1, wherein the elongate member comprises a tubular member defining a lumen sized to receive the medical device therein.

11. The system of claim 10, wherein the tubular member comprises a longitudinal slit extending between proximal and distal ends of the tubular member and defining opposing longitudinal edges.

12. The system of claim 11, wherein the longitudinal edges are separable to accommodate inserting the medical device laterally between the longitudinal edges such that the medical device is captured within the lumen.

13-16. (canceled)

17. The system of claim 1, wherein the expandable filter is compressible to a collapsed condition sized for introduction into the body lumen, and is resiliently biased to expand towards an enlarged condition when deployed within the body lumen.

18. The system of claim 17, wherein the expandable filter defines an open end and a closed end in the enlarged condition.

19. The system of claim 18, wherein the medical device comprises a catheter including a diagnostic or treatment element on the distal end, and wherein the open end is disposed closer to the diagnostic or treatment element than the closed end when the filter device is mounted onto the catheter.

20. The system of claim 19, wherein the diagnostic or treatment element comprises a balloon.

21. A method for performing a procedure at a target location within a patient's body, comprising:
providing an elongate medical device including proximal and distal ends and one or more diagnostic or treatment elements on the distal end;
mounting a filter device on the medical device such that the filter device is substantially fixed axially relative to the medical device and an expandable filter of the filter device is disposed proximal to the one or more elements;
introducing the medical device together with the filter device with the expandable filter in a collapsed condition into the patient's body until the distal end is disposed at a target location within a body lumen;
deploying the expandable filter such that the expandable filter expands to an enlarged condition at the target location; and
performing a procedure at the target location using the one or more elements on the medical device, the expanded expandable filter capturing material released during the procedure.

22. The method of claim 21, further comprising:
collapsing the expandable filter towards the collapsed condition; and
removing the filter device and medical device substantially simultaneously from the target location, thereby removing the captured material within the collapsed expandable filter.

23-26. (canceled)

27. The method of claim 21, wherein the medical device comprises a catheter, and the one or more elements comprise at least one of a balloon, a stent, and a prosthetic valve.

28-30. (canceled)

31. A method for performing a procedure at a target location within a patient's body, comprising:
providing an elongate medical device including proximal and distal ends and one or more diagnostic or treatment elements on the distal end;
introducing the distal end of the medical device into the patient's body until the distal end is disposed at a target location within a body lumen;
advancing a filter device over the medical device until an expandable filter on the filter device is disposed proximal to the one or more elements within the target location;
deploying the filter such that the filter expands to an enlarged condition at the target location;
performing a procedure at the target location using the one or more elements on the medical device, the filter capturing material released during the procedure;
removing the distal end of the medical device from the target location, while leaving the filter device with the filter deployed at the target location; and
introducing the distal end of a second medical device into the patient's body until the distal end is disposed at the target location.

32-40. (canceled)

41. A filter device, comprising:
an elongate member including a proximal end, a distal end sized for introduction into a patient's body;
an expandable filter on the distal end of the elongate member; and
one or more features on the elongate member for engaging an outer surface of a catheter or sheath to mount and substantially fix axially the filter device onto the catheter or sheath.

42. (canceled)

43. The filter device of claim 41, wherein the one or more features comprise a collar on the distal end of the elongate member adjacent the expandable filter sized to at least partially surround and frictionally engage the outer surface of the catheter or sheath.

44. The filter device of claim 43, further comprising one or more additional collars on the elongate member spaced apart proximally from the collar on the distal end.

45-69. (canceled)

70. The system of claim 1, wherein the elongate member has a length that is substantially shorter than a length of the medical device.

71. The system of claim 1, wherein the medical device is a catheter or sheath.

72. The system of claim 1, wherein the one or more features comprise material that frictionally engages or provides an interference fit with the outer surface of the medical device to substantially fix the filter device axially relative to the medical device.

73. The system of claim 1, wherein the one or more features allow the filter device to be separated from the medical device.

74. The method of claim 21, wherein the medical device is a guide catheter or sheath.
